(19) United States
(12) Patent Application Publication
(10) Pub. No.: US 2021/0246144 A1
(43) Pub. Date: Aug. 12, 2021

(54) A SET OF MITOCHONDRIA-TARGETED COMPOUNDS

(71) Applicant: MITOTECH S.A., Luxembourg (LU)

(72) Inventors: Maxim V. Skulachev, Moscow (RU); Galina A. Korshunova, Moscow (RU); Anna V. Shiskina, Moscow (RU); Anton Petrov, Woodland Hills, CA (US)

(73) Assignee: MITOTECH S.A., Luxembourg (LU)

(21) Appl. No.: 16/625,575

(22) PCT Filed: Jun. 19, 2018
(86) PCT No.: PCT/IB2018/000773

§ 371 Date: Dec. 20, 2019

Related U.S. Application Data
(60) Provisional application No. 62/524,782, filed on Jun. 26, 2017.

(51) Int. Cl. C07D 491/147; C07D 311/78; C07D 271/12

(52) U.S. Cl. CPC C07D 491/147; C07D 271/12; C07D 311/78

(57) ABSTRACT

Here are described SkQ compounds containing cations of various types: alkyl(triphenyl)phosphonium cation, quaternary ammonium cations, including pH-dependent and permanent cations of rhodamines, berberine and palmatine alkaloids.

### FIELD OF THE INVENTION

This invention relates to the fields of pharmaceutics and medicine, and, in particular, concerns design and synthesis mitochondrially targeted compounds including mitochondrially targeted antioxidants.

### BACKGROUND OF THE INVENTION

Several compounds of mitochondrially targeted antioxidants family were previously disclosed in WO9926954 and related patent applications, WO2007046729, WO2011059355 and WO2015063553. Numerous studies showed useful biological activity of these compounds in experimental models of different pathologies and in several clinical trials (Lukashev et al, 2014, Prog Mol Biol Transl Sci., 127:251-65 and Skulachev et al, 2011, Curr Drug Targets. 12(6):800-26). Different mitochondrially targeted antioxidants vary in their chemical and biological properties such as stability, efficacy, pharmacokinetics etc. Thus development, design and synthesis of new examples of these compounds are important tasks for modern pharmacology. Generally mitochondrially targeted antioxidants consist of an antioxidant moiety, a linker, and a lipophilic cation moiety responsible for mitochondrial targeting. Mitochondrially targeted antioxidants with plastoquinone derivatives as antioxidant moiety were termed SkQ compounds or SkQs. Same approach can be applied for construction of other mitochondrially targeted compounds (for example mitochondrially targeted uncouplers or fluorescent dyes).

### BRIEF SUMMARY OF THE INVENTION

Here are described SkQ compounds containing cations of various types: alkyl(triphenyl)phosphonium cation, quaternary ammonium cations, including pH-dependent and permanent cations of rhodamines, berberine and palmatine alkaloids. The synthesis of mitochondria-targeted uncouplers of oxidative phosphorylation based on fluorescent dyes is also described.

### BRIEF DESCRIPTION OF THE DRAWINGS

FIG. 1 shows the structural formulas of plastoquinone containing rhodamine derivatives (25-27).

FIG. 2 shows the structural formulas of n-alkyl esters of rhodamine 19 (28, 29), rhodamine 110 (30, 31), rhodamine B (32-35), and rhodamine 101 (36).

FIG. 3 shows the structural formulas of conjugates of palmatine with plastoquinone via various linkers (44-46).

FIG. 4 shows the structural formulas of n-alkyl esters of berberine (47-50) and palmatine (51-54).

FIG. 5 shows the structural formulas of mitochondria-targeted derivatives of the dyes eosine Y (66) and brilliant rose (67).

### DETAILED DESCRIPTION OF THE PREFERRED EMBODIMENTS

The following abbreviations are used in the examples. MitoQ—[10-(4,5-dimethoxy-2-methyl-3,6-dioxocyclohexa-1,4-dien-1-yl)decyl](triphenyl)phosphonium bromide; SkQ1-[10-(4,5-dimethyl-3,6-dioxocyclohexa-1,4-dien-1-yl)decyl](triphenyl)phosphonium bromide; ROS—reactive oxygen species; HPLC—high pressure liquid chromatography; NMR—nuclear magnetic resonance; SkQ3-[10-(2,4,5-trimethyl-3,6-dioxocyclohexa-1,4-dien-1-yl)decyl](triphenyl)phosphonium; SkQT-p—[10-(4-methyl-3,6-dioxocyclohexa-1,4-dien-1-yl)decyl](triphenyl)phosphonium; SkQT-m—10-(5-methyl-3,6-dioxocyclohexa-1,4-dien-1-yl)decyl](triphenyl)phosphonium; SkQR1—(N-[(3Z)-9-[2-({[10-(4,5-dimethyl-3,6-dioxocyclohexa-1,4-dien-1-yl)decyl]oxy}carbonyl)phenyl]-6-(ethylamino)-2,7-dimethyl-3H-xanten-3-yliden]ethanaminium chloride; DMF—dimethylformamide; NBD—7-nitro-2,1,3-benzoxadiazole.

The invention consists on the following experimental examples providing ways of synthesis and use of novel mitochondrially targeted compounds.

The invention provides data on the design and synthesis of new biologically active compounds—mitochondria-targeted antioxidants that are natural (or synthetic) p-benzoquinones, conjugated via lipophilic linker with (triphenyl)phosphonium or ammonium cations with a delocalized charge. Also described is the synthesis of mitochondria-targeted antioxidants—uncouplers of oxidative phosphorylation-based on the fluorescent dyes.

The following examples are intended to further illustrate certain embodiments of the invention, and are not to be construed to limit the scope of the invention.

### Example 1

### Synthesis of SkQ Compounds Containing Ammonium Cations

SkQ-compounds containing rhodamines. Rhodamines are a class of fluorescent compounds—xanthene dyes derivatives; their advantage is in a simple tracking of their penetration into mitochondria, into individual cells and into whole organisms. All of them contain in their structure ammonium nitrogen (in the form of pH-dependent or permanent nitrogenous cations), which can be used in the construction of Skulachev ions to form a quaternary ammonium cation. We used rhodamine 19 (RI), rhodamine 110, rhodamine B and rhodamine 101 as the starting materials. The free carboxyl group presented in them proved to be convenient for attaching the antioxidant moiety through an ester bond. For this, a cesium salt of rhodamine was preliminarily prepared, and then condensed with the plastoquinone bromodecyl derivative 2. Scheme XI reflects the synthesis of a mitochondria-targeted cation based on rhodamine 19, namely, (N-[(3Z)-9-[2-({[10-(4,5-dimethyl-3,6-dioxocyclohexa-1,4-dien-1-yl)decyl]oxy} carbonyl)phenyl]-6-(ethylamino)-2,7-dimethyl-3H-xanthen-3-ylidene]ethanaminium chloride (23). For this compound, the abbreviated name SkQR1 further is used.

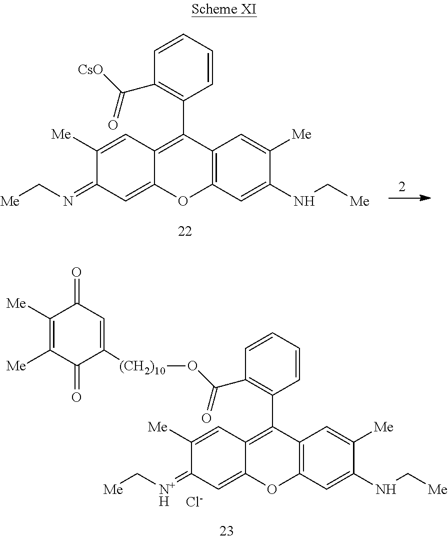

The cesium salt of rhodamine 19 (22) was prepared with a fourfold excess of cesium carbonate in boiling methanol, after the reaction mixture cooled, the salt was isolated by filtration; the yield was 80%. When the cesium salt 22 with the derivative 2 was heated at 50-60° C. in dimethylformamide (DMF), the conjugate SkQR1 of quinone 1 and rhodamine was formed. Its isolation and purification were carried out by silica gel column chromatography in a chloroform-methanol system (4:1). For greater storage stability, the substance was converted into the hydrochloride form—it was treated with a 4M solution of hydrogen chloride in absolute dioxane, followed by evaporation and drying. Thereby SkQR1 was obtained as hydrochloride in 65% yield.

For SkQR1, as well as for SkQ1, the greatest amount of biological data was collected.

The SkQR1 analogue differs from it in the toluquinone moiety presented instead of plastoquinone in the structure. Starting from the toluquinone derivative p-isomer 7a and the rhodamine cesium salt 22, SkQTR1: 10-(4-methyl-3,6-dioxocyclohexa-1,4-dien-1-yl)decyl-2-[(3Z)-6-(ethylamino)-3-(ethylimino)-2,7-dimethyl-3H-xanthen-9-yl] benzoate (24) as shown in Scheme XII.

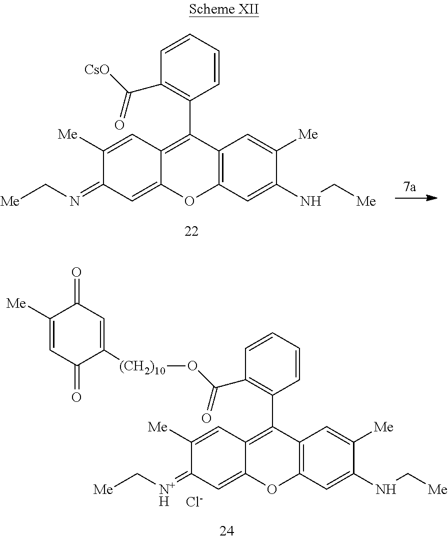

The desired product was isolated by column chromatography on silica with dichloromethane:ethanol (5:1) and subsequent purification by reversed-phase HPLC.

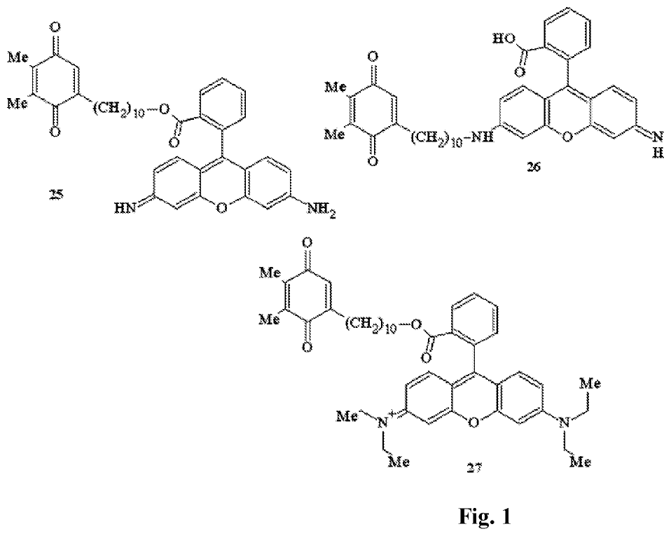

Under analogous conditions, 10-(4,5-dimethyl-3,6-dioxo-cyclohexa-1,4-dien-1-yl)decyl 2-(6-amino-3-imino-3H-xanthen-9-yl)benzoate (25) was synthesized from rhodamine 110 cesium salt and bromodecylplastoquinone 2. If the free rhodamine 110 was reacted in pyridine at 80° C., the main reaction product was 2-(6-{[10-(4,5-dimethyl-3,6-dioxocyclohexa-1,4-dien-1-yl)decyl]amino}-3-imino-3H-xanthen-9-yl) benzoic acid (26). In the case of rhodamine B, an analogue, 6-(diethylamino)-9-[2-({[10-(4,5-dimethyl-3,6-dioxocyclohexa-1,4-dien-1-yl)decyl]oxy}carbonyl)phenyl]-N,N-diethyl-3H-xanthene-3-iminium (27) was prepared (FIG. 1).

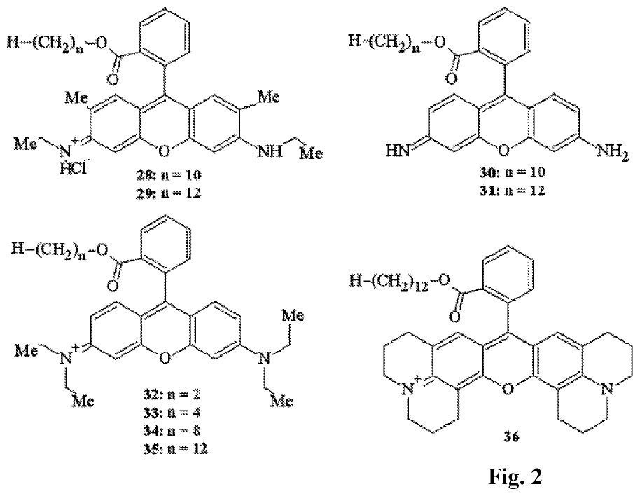

The rhodamine derivatives not bearing an antioxidant fragment were synthesized as bromides (chlorides or iodides) (FIG. 2) for the control in biological experiments, in particular n-decyl and n-dodecyl esters of rhodamine 19, respectively: decyl 2-[(3E)-6-(ethylamino)-3-(ethylimino)-2,7-dimethyl-3H-xanthen-9-yl]benzoate (28) and dodecyl 2-[(3E)-6-(ethylamino)-3-(ethylimino)-2,7-dimethyl-3H-xanthen-9-yl]benzoate (29) (FIG. 2). The esterification of rhodamine 110 with an appropriate aliphatic alcohol in the presence of sulfuric acid led to n-decyl rhodamine ester-decyl 2-(6-amino-3-imino-3H-xanthen-9-yl)benzoate (30) and n-dodecyl rhodamine ester-dodecyl 2-(6-amino-3-imino-3H-xanthen-9-yl)benzoate (31).

Compounds 28 and 29, as well as SkQR1, are soft cationic mitochondrial uncouplers (protonophores), capable of causing a decrease in the mitochondrial membrane potential, and, as a consequence, reduction of ROS production by mitochondria, due to the dissociating protons of the ammonium rhodamine atom.

Rhodamines B and 101, unlike rhodamine 19, contain in their structure a quaternary ammonium cation with a constant charge, hence the protonation process is hindered and the protonophoric activity is exhibited. The esterification of rhodamine B with alcohols in an acidic medium gave a series of rhodamine B alkyl esters with the common name 9-{2-[(alkyloxy)carbonyl]phenyl}-6-(diethylamino)-N,N-diethyl-3H-xanthene-3-iminium, where “alkyl” means “ethyl” in the case of compound 32, “butyl”—33, “octyl”—34, “dodecyl”—35 (FIG. 2) The n-dodecyl ester of rhodamine 101—16-{2-[(dodecyloxy)carbonyl]phenyl}-3-oxa-9λ⁵,23-diazaheptacyclo[17.7.1.1^{5.9}0^{2.1}.0^{4.15}0^{23.27}.0^{13.28}]octacosa-1(27),2(17),4,9(28),13,15,18-heptane-9-ylium (36) (FIG. 2). Cations based on rhodamine B esters have been studied in view of the hydrocarbon chain length influence on diffusion through lipid membranes.

SkQ-compounds containing berberine and palmatine. A fairly promising group of compounds are the derivatives of berberine and palmatine, belonging to the family of isoquinoline alkaloids contained in plants of the Barberry families, and possess a variety of pharmacological properties, including antimicrobial and cytotoxic activities. These features, as well as the presence in the structure of a permanent quaternary ammonium ion, attracted our attention in terms of their use as a basis for the construction of mitochondria-targeted antioxidants.

Attempts to introduce the hydrocarbon linker directly into the ring of the molecule through its bromination, followed by the cross-coupling reactions, ultimately proved unsuccessful. Therefore, it was decided to modify berberine with a carboxymethyl group introduced at position 13 of the heterocyclic molecule. Synthesis of 13-substituted derivatives of berberine with this modification is described in the literature. We reproduced the synthesis of the modified compound (Scheme XIII) by reduction of berberine (37) to dihydroberberine (38), alkylation of the latter with ethyl bromoacetate and subsequent reduction by sodium borohydride to give tetrahydroberberine (39).

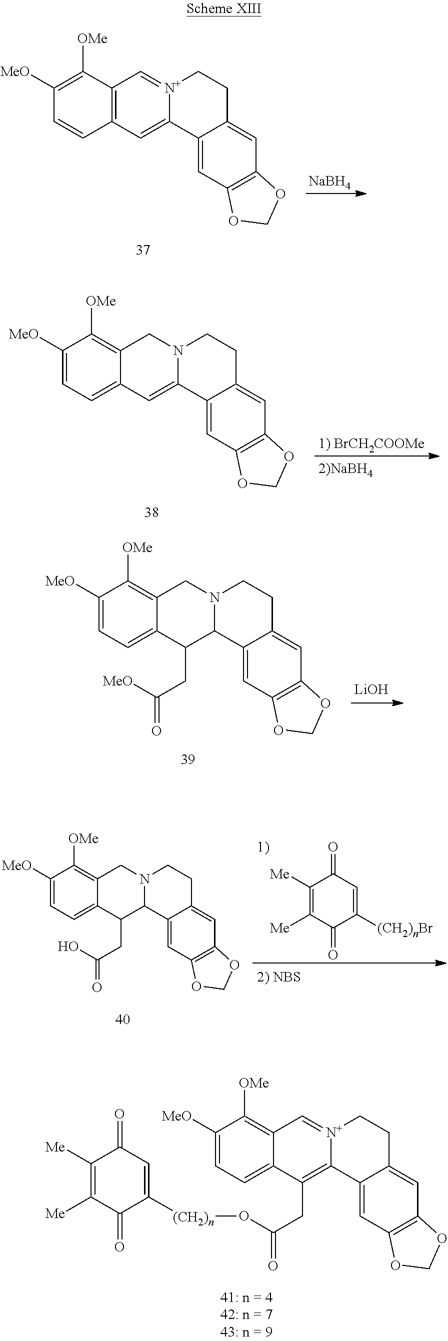

By gentle hydrolysis of 39 a key compound was obtained—2-{16,17-dimethoxy-5,7-dioxa-13-azapentacyclo[11.8.0.0^{2.14}.0^{4.8}.0^{15.00}]henicosa-2,4(8),9,15,17,19-hexaen-21-yl}acetic acid (40), which we further used to prepare a series of berberine conjugates with plastoquinone via hydrocarbon linkers of different lengths. The cesium salt of acid 40 was condensed with preformed bromoalkyl plastoquinone derivatives (where “alkyl” is “butyl”, “heptyl” and “nonyl”), as it was described for compound 2, and after oxidation of the condensation products with N-bromosuccinimide in chloroform, the desired compounds 21-(2-{[4-(4,5-dimethyl-3,6-dioxocyclohexa-1,4-dien-1-yl)butyl]oxy}-2-oxoethyl)-16,17-dimethoxy-5,7-dioxa-13,λ⁵-azapentacyclo[11.8.0.0^{21.0}.0^{4.8}.0^{15.20}]henicosa-1(21),2,4(8),9,13,15(20),16,18-octaen-13-ylium (41), 21-(2-{[7-(4,5-dimethyl-3,6-dioxocyclohexa-1,4-dien-1-yl)heptyl]oxy}-2-oxoethyl)-16,17-dimetoxy-5,7-dioxa-13λ⁵-azapentacyclo[11.8.0.0^{2.10}.0^{48.0}.0^{15.20}]henicosa-1(21),2,4(8),9,13,15(20),16,18-octaen-13-ylium (42) and 21-(2-{[9-(4,5-dimethyl-3,6-dioxocyclohexa-1,4-dien-1-yl)nonyl]oxy}-2-oxoethyl)-16,17-dimethoxy-5,7-dioxa-13λ⁵-azapentacyclo[11.8.0.0^{21.0}.0^{4.8}.0^{15.20}]henicosa-1(21),2,4(8),9,13,15(20),16,18-octaen-13-ylium (43)—as the most promising it was abbreviated SkQBerb.

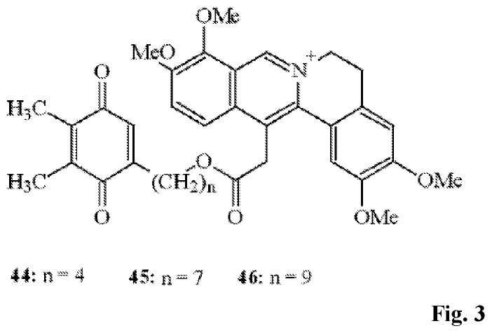

Under similar conditions, palmatine conjugates (in the form of bromides) were prepared: 13-(2-{[4-(4,5-dimethyl-3,6-dioxocyclohexa-1,4-dien-1-yl)butyl]oxy}-2-oxoethyl)-3,4,10,11-tetramethoxy-7,8-dihydro-6λ⁵-azatetraphen-6-ylium (44), 13-(2-{[7-(4,5-dimethyl-3,6-dioxocyclohexa-1,4-dien-1-yl)heptyl]oxy}-2-oxoethyl)-3,4,10,11-tetramethoxy-7,8-dihydro-6λ⁵-azatetraphen-6-ylium (45), 13-(2-{[9-(4,5-dimethyl-3,6-dioxocyclohexa-1,4-dien-1-yl)nonyl]oxy}-2-oxoethyl)-3,4,10,11-tetramethoxy-7,8-dihydro-6λ⁵-azatetraphen-6-ylium (46) shown in FIG. 3. The latter is abbreviated SkQPalm.

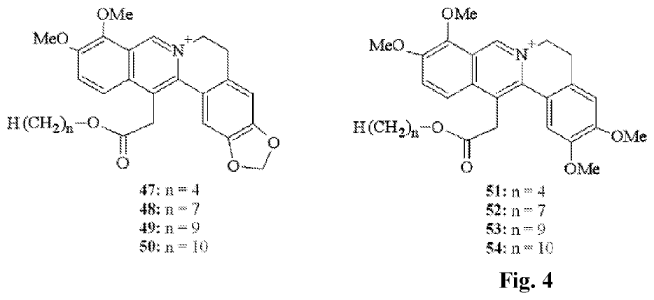

SkQBerb analogs lacking a plastoquinone moiety were prepared as control samples: 16,17-dimethoxy-21-[2-(butyloxy)-2-oxoethyl]-5,7-dioxa-13λ⁵-azapentacyclo[11.8.0.0^{21.0}.0^{4.8}.0^{15.20}]henicosa-1(21),2,4(8),9,13,15(20),16,18-octaen-13-ylium (47), 16,17-dimethoxy-21-[2-(heptyloxy)-2-oxoethyl]-5,7-dioxa-13λ⁵-azapentacyclo[11.8.0.0^{21.0}.0^{4.8}.0^{15.20}]henicosa-1(21),2,4(8),9,13,15 (20),16,18-octaen-13-ylium (48), 16,17-dimethoxy-21-[2-(nonyloxy)-2-oxoethyl]-5,7-dioxa-13λ⁵-azapentacyclo[11.8.0.0^{21.0}.0^{4.8}.0^{15.20}]henicosa-1(21),2,4(8),9,13,15(20),16,18-octaen-13-ylium (49), 16,17-dimethoxy-21-[2-(decyloxy)-2-oxoethyl]-5,7-dioxa-13λ⁵-azapentacyclo[11.8.0.0^{21.0}.0^{4.8}.0^{15.20}]henicosa-1(21),2,4(8),9,13,15(20),16,18-octaen-13-ylium (50) and SkQPalm analogues without plastoquinone fragment: 3,4,10,11-tetramethoxy-13-[2-(butyloxy)-2-oxoethyl]-7,8-dihydro-6λ⁵-azatetraphen-6-ylium (51), 3,4,10,11-tetramethoxy-13-[2-(heptyloxy)-2-oxoethyl]-7,8-dihydro-6λ⁵-azatetraphen-6-ylium (52), 3,4,10,11-tetramethoxy-13-[2-(nonyloxy)-2-oxoethyl]-7,8-dihydro-6λ⁵-azatetraphen-6-ylium (53) and 3,4,10,11-tetramethoxy-13-[2-(decyloxy)-2-oxoethyl]-7,8-dihydro-6λ⁵-azatetraphen-6-ylium (54) (FIG. 4).

The alkyl esters of berberine 47-50 (and palmatine 51-54) were obtained by reacting of the corresponding n-bromoalkanes with cesium salt of modified berberine 40 (or palmatine) under heating (70-80° C.) for two days or by condensation 40 with the corresponding alcohol on cooling in the presence of N,N′-dicyclohexylcarbodiimide.

Among the ammonium cations, the SkQ analogs bearing berberine and palmatine are of greatest interest. It was established that SkQBerb and SkQPalm are not inferior in properties to SkQ1. They penetrate through bilayer phospholipid membranes, accumulate in isolated mitochondria or live cultures of human cells, inhibit lipid peroxidation in isolated mitochondria in nanomolar concentrations; and their prooxidant effect is manifested at significantly higher concentrations [64, 65].

### Example 2

### Synthesis of Mitochondria-Targeted Fluorescent Uncouplers

Derivatives of fluorescein and its analogues. Fluorescein and its derivatives are widely applied in scientific and medical practice. It is known that the carboxy-group of fluorescein does not play an important role in functional features of the compound, therefore it can be modified to provide the desired properties. The reaction was accomplished by converting fluorescein (55) to the cesium salt (56) by heating with cesium carbonate in DMF, and further condensation with alkyl bromide under prolonged heating in DMF (Scheme XIV).

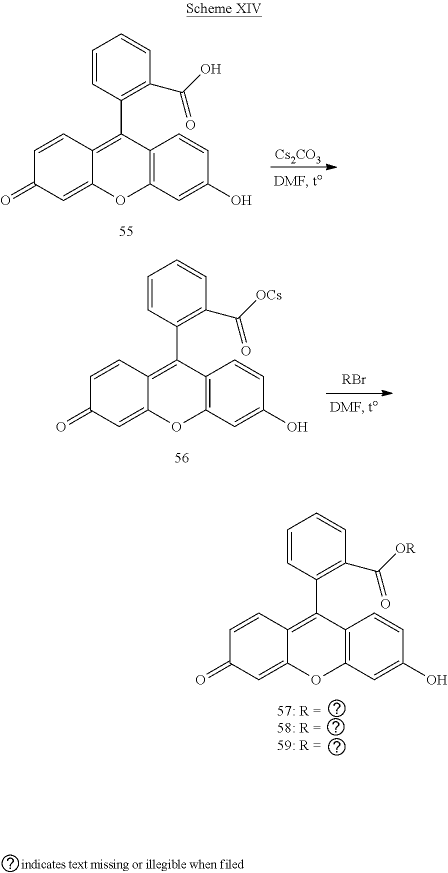

n-Butyl, n-octyl and n-dodecyl esters of fluorescein have been synthesized, increasing the hydrophobicity of the latter and enhancing its solubility in a nonpolar medium: butyl 2-(6-hydroxy-3-oxo-3H-xanthen-9-yl)benzoate (57), octyl-2-(6-hydroxy-3-oxo-3H-xanthen-9-yl)benzoate (58) and dodecyl 2-(6-hydroxy-3-oxo-3H-xanthen-9-yl)benzoate (59). Under such conditions, along with the desired product, derivatives of the hydroxyl group were also obtained.

Esterification of fluorescein with an appropriate alcohol in the presence of sulfuric acid, as described in Brown et al., proceeds selectively on the carboxyl group to form esters 57-59.

Conjugation of bromoalkyl(triphenyl)phosphonium cations with fluorescein led to the creation of fluorescent mitochondria-targeted protonophoric uncouplers—potential therapeutic agents for the oxidative stress diseases treatment. Scheme XV displays the synthesis of these conjugates.

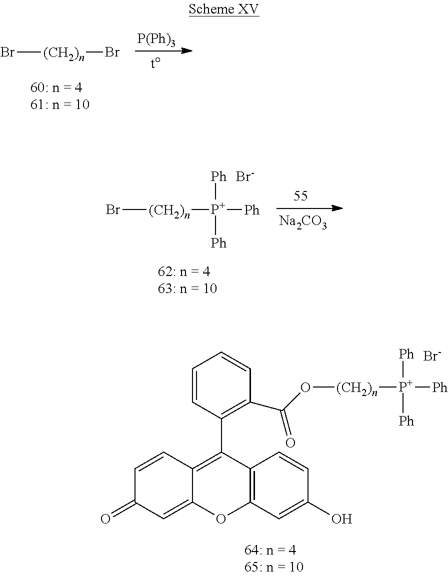

Dibromoalkanes (60, 61) were heated with (triphenyl)phosphine in benzene at 80° C., and the obtained bromoalkyl(triphenyl)phosphonium bromides (62, 63) were reacted with fluorescein in the presence of sodium carbonate. As a result, bromides of {4-[2-(6-hydroxy-3-oxo-3H-xanthen-9-yl)benzoyloxy]butyl}(triphenyl)phosphonium (64) and {10-[2-(6-hydroxy-3-oxo-3H-xanthen-9-yl)benzoyloxy]decyl}(triphenyl)phosphonium (65). These compounds are fluorescent uncouplers possessing neuroprotective and nephroprotective properties.

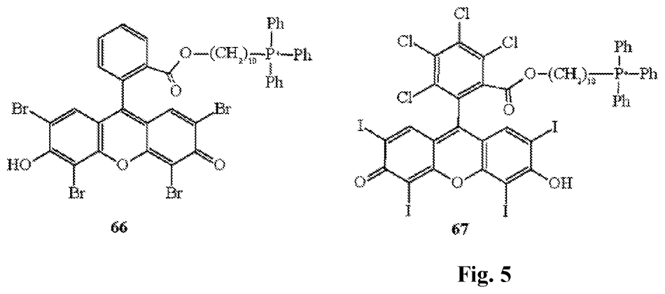

In addition, analogous derivatives with eosin Y—triphenyl({10-[2-(2,4,5,7-tetrabromo-6-hydroxy-3-oxo-3H-xanthen-9-yl)benzoyloxy]decyl})phosphonium (66) and with brilliant rose dye—triphenyl({10-[2,3,4,5-tetrachloro-6-(6-hydroxy-2,4,5,7-tetraiodo-3-oxo-3H-xanthene-9-yl)benzoyloxy]decyl})phosphonium (67) were obtained (FIG. 5).

Derivatives of 7-nitro-2,1,3-benzoxadiazole. We synthesized a large series of fluorescent uncouplers based on the derivatives of 7-nitro-2,1,3-benzoxadiazole (NBD), which has a high quantum yield of fluorescence. The reaction of 4-chloro-7-nitro-2,1,3-benzoxadiazole (NBD-Cl) with the corresponding alkylamines yielded 7-nitro-N-alkyl-2,1,3-benzoxadiazole-4-amines with different alkyl substituent lengths of the common formula H—(CH₂)ₙ—NH-NBD, where n=8, 9, 10 and 12 (68-71) (Scheme XVI).

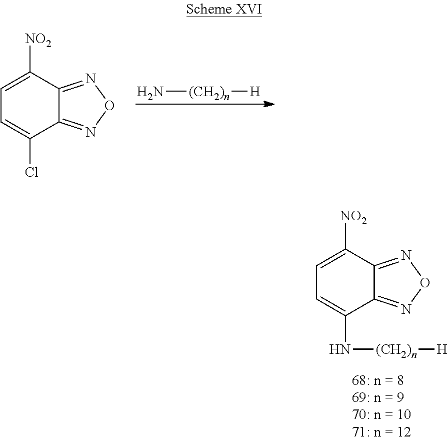

The reaction proceeded in absolute chloroform in the presence of sodium carbonate at room temperature. The reaction progress was observed by the appearance of a fluorescent spot on the chromatogram. The initial NBD-Cl is a fluorogenic substance, i.e. it forms a fluorescent product. The target compounds were purified by silica gel column chromatography with dichloromethane as the eluent. The yield of final compounds comprised 55-65%. Derivatives bearing the alkyl substituent of 8-12 carbon atoms are optimal for the ability to bind lipid membranes.

It was revealed that compounds 68-71 exhibit protonophore activity in liposomes and uncoupling activity in mitochondria. Inclusion of an alkyl chain of a certain length compensates for the deterioration of the uncoupling ability of NBD due to its high pKa value. It was previously shown that the introduction of an aryl substituent in 4-amino-NBD shifts pKa to neutral values. Therefore, it was interesting to obtain a series of aryl derivatives of NBD—7-nitro-N-(4-alkylphenyl)-2,1,3-benzoxadiazole-4-amines (Scheme XVII) of the general formula H—(CH₂)ₙ-Ph-NBD, where n=0, 1, 4, 6 (72-75).

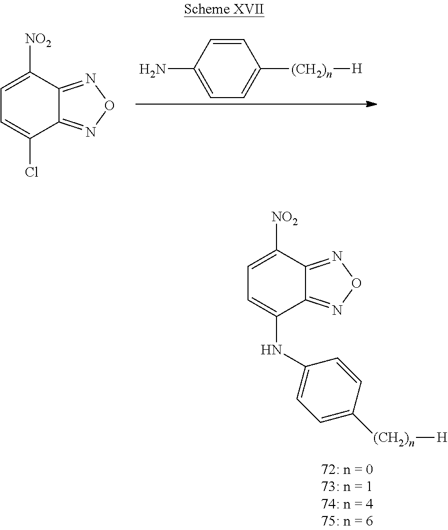

1. A mitochondrially targeted compound having rhodamine derivative as mitochondria targeting moiety selected from group consisting of

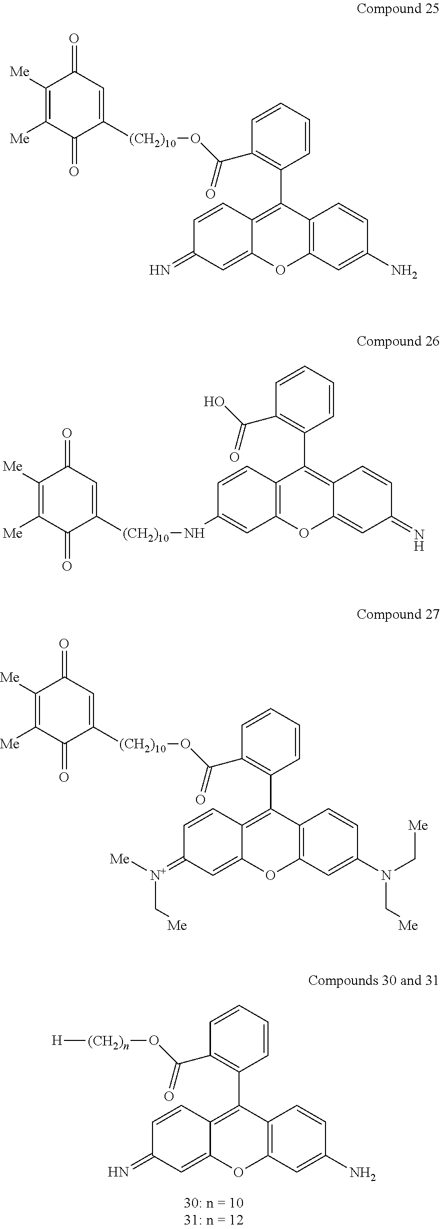

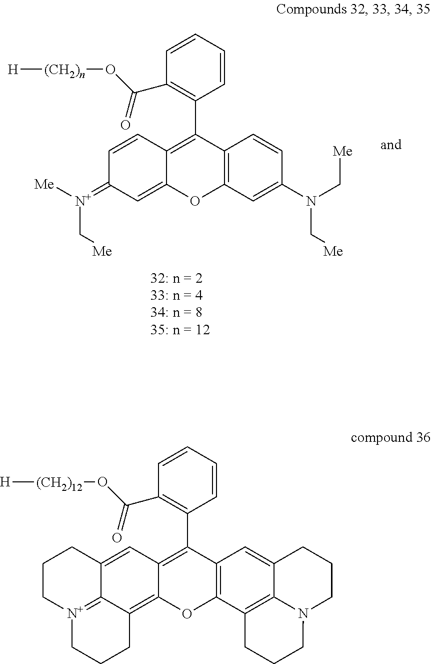

2. A mitochondrially targeted compound having berberin or palamatin derivative as mitochondria targeting moiety selected from the group consisting of Compounds 49 and 50:

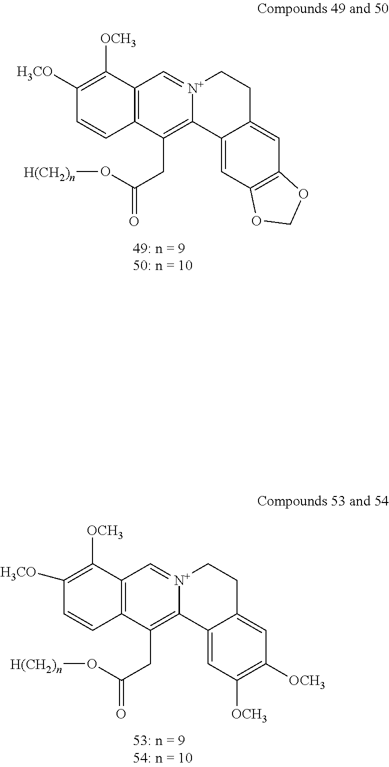

3. A fluorescein derivative selected from the group consisting of:

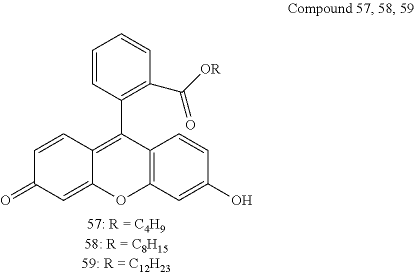

4. A fluorescein or its analog derivative linked to triphenylphosphonium moiety selected from the following group of compounds:

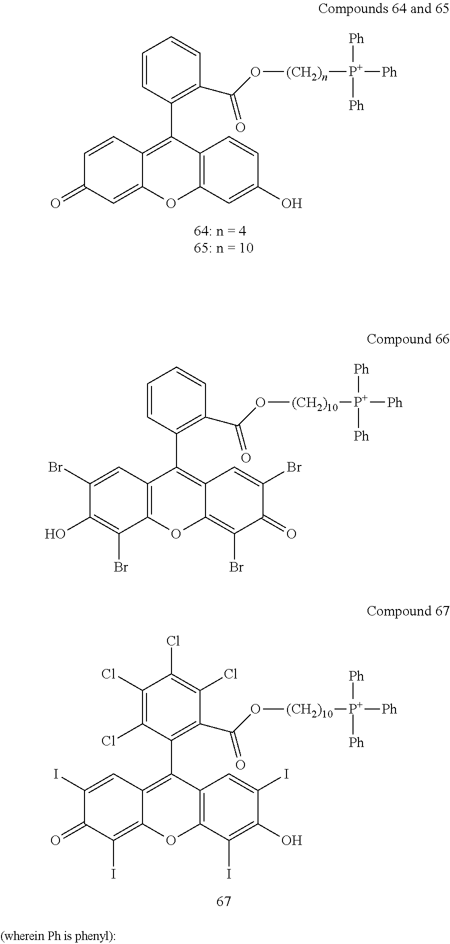

5. A derivative of of 7-nitro-2,1,3-benzoxadiazole selected from the following group of compounds: [chemical structure]
